(19) United States
(12) Patent Application Publication
(10) Pub. No.: US 2023/0201138 A1
(43) Pub. Date: Jun. 29, 2023

(54) TOPICAL PHARMACEUTICAL COMPOSITION COMPRISING AT LEAST AMITRIPTYLINE, FOR THE TREATMENT OF PERIPHERAL NEUROPATHIC PAIN

(71) Applicant: ALGOTHERAPEUTIX, Suresnes (FR)

(72) Inventor: Céline Gréco, Paris (FR)

(73) Assignee: ALGOTHERAPEUTIX, Suresnes (FR)

(21) Appl. No.: 18/170,804

(22) Filed: Feb. 17, 2023

Related U.S. Application Data
(63) Continuation of application No. 16/608,783, filed on Oct. 25, 2019.

(30) Foreign Application Priority Data
Apr. 25, 2017 (FR) 1753577

(51) Int. Cl. A61K 31/135; A61P 25/02; A61K 47/06; A61K 47/30; A61K 47/44; A61K 9/10; A61K 9/06; A61K 45/06

(52) U.S. Cl. CPC A61K 31/135; A61P 25/02; A61K 47/06; A61K 47/30; A61K 47/44; A61K 9/10; A61K 9/06; A61K 45/06

(57) ABSTRACT

A method of applying a topical treatment of chemotherapy-induced peripheral neuropathic pain with a pharmaceutical composition comprising from 10% to 30% by weight of amitriptyline or of a pharmaceutically acceptable salt or ester thereof, relative to the total weight of the composition, amitriptyline, for use in the topical treatment of.

### CROSS-REFERENCE TO RELATED APPLICATION

This application is a continuation of application Ser. No. 16/608,783, filed Oct. 25, 2019, which is a U.S. National Stage of PCT/EP2018/059948, filed Apr. 18, 2018, which claims the benefit of French Application No. 1753577, filed Apr. 25, 2017, the entire disclosures of said applications are hereby incorporated by reference herein.

### BACKGROUND

The present invention relates to a pharmaceutical composition comprising from 10% to 30% by weight, relative to the total weight of the composition, of amitriptyline or of a pharmaceutically acceptable salt thereof, for use in the topical treatment of chemotherapy-induced peripheral neuropathic pain.

Peripheral neuropathic pain is caused by damage to nerve structures such as peripheral nerve endings or nociceptors which become extremely sensitive to stimulation and which can generate pulses in the absence of stimulation.

This damage can occur for many reasons, such as trauma, diseases such as diabetes, shingles and advanced-stage cancers, chemotherapy treatments or else a chemical burn. The lesion of the peripheral nerve can result in pathological states characterized by the presence of continuous spontaneous pain which is superficial (sensation of painful burning or cold) or deep (compression or vice sensation), paroxysmal pain (electrical discharges, stabbing) with, upon clinical examination, hypoesthesia or, on the contrary, hyperalgesia (increased response to harmful stimuli), allodynia (pain induced by a non-painful stimulus) or else hyperpathia (persistent pain during normally non-nociceptive repeated stimulations). Neuropathies may also be associated with sensory signs such as paresthesia, numbness, pruritis.

Chemically induced neuropathies are particularly frequent, disabling and difficult to treat. They are dose-dependent. Peripheral nerve damage represents the majority of neurological damage associated with chemotherapy toxicity. It is the consequence of direct toxic damage to the axon or of demyelination and represents the most frequent limiting factor after haematological toxicity.

Thus, before chemically induced neuropathies occur, the chemotherapy doses will be reduced or the treatment may even be stopped, thus constituting an actual reduction in the patient's chances.

Thus, it has been possible to observe neuropathies subsequent to treatment with alkaloids (vincristine, vinblastine, vinorelbine) often leading to small-fibre damage, platinum derivatives (oxaliplatin, cisplatin, carboplatin), anti-topoisomerases (VP16), proteasome inhibitors (bortezomib, carfilzomib), thalidomide derivatives such as lenalidomide, taxanes such as taxol or taxotere which instead affect large fibres. There are also neuropathies after treatment by immunotherapy, such as for example anti-CD20, anti-CD30, anti-CD38.

This chemically induced pain occurs according to poorly understood mechanisms, thus certain authors think that it is due to a direct toxic effect on the sensory axon, to demyelination or else to an impairment of calcium metabolism, associated with damage to mitochondria, the site of action of paclitaxel and of vincristine, for example.

Thus, it is known that taxanes intervene on the spinal ganglion, the microtubules, the mitochondria and the nerve endings, platinum salts intervene on myelin and ion channels, while alkaloids intervene on myelin and microtubules.

This neuropathic pain is often refractory to the usual analgesic treatments and leads to decreases in doses or even interruptions of chemotherapy. It is at the current time treated with per os treatments comprising antidepressants (Amitriptyline, Duloxetine, Venlafaxine, etc.) and/or antiepileptics (Gabapentin, Pregabalin). Unfortunately, these systemic treatments induce major side effects (dizziness, drowsiness, memory loss, dryness of the mouth or even urine retention, nausea, etc.) leading to poor treatment adherence and pain control which is not very satisfactory.

This pain mostly affects the extremities of the hands and feet and leads to a considerable impairment of the quality of life of patients with a functional impotence that can range up to the inability to walk, gripping difficulties, impaired sleep, the occurrence of a depressive syndrome or even a suicidal tendency. The impact on the social and professional life can also be very significant.

The intensity of the pain is often described as severe with patients who evaluate their pain at more than 7/10 on the visual analogue scale (pain graded from 0 to 10).

Chemotherapy-induced neuropathic pain is essentially toxic in origin, as recalled above, whereas post-shingles neuropathies are generally linked to nerve damage owing to a prior infection by the herpes zoster virus. The damaged nerves are no longer capable of correctly transmitting the signals from the skin to the brain. Tricyclic antidepressants are chemical compounds discovered at the beginning of the 1950s. They are widely used to treat various psychiatric disorders, in particular depression, panic disorders, obsessive compulsive disorders, enuresis in children, bipolar disorders and hyperactivity. They are also used as analgesics.

These compounds are generally administered orally.

Amitriptyline is a tricyclic antidepressant discovered in 1960, which has frequently been recommended as first-line treatment for major depression, post-traumatic stress disorder (PTSD), generalized anxiety disorder (GAD), social phobia (SP), panic disorder, fibromyalgia, chronic musculoskeletal pain, akinesia in Parkinson's disease, cataplexy, migraines, Parkinson's disease, vasomotor symptoms of the menopause, nocturnal enuresis, premenstrual dysphoric disorder (PMDD), bipolar disorder, bulimia, obsessive compulsive disorders (OCD) and neuropathic pain.

In the past, patients were generally treated by administering analgesics to relieve the pain. The oral route was widely preferred.

However, the oral administration of amitriptyline, as for all tricyclic antidepressants, has many side effects linked to their anti-cholinergic effects (risk of arterial hypotension, sinus tachycardia or supraventricular tachycardia, in rare cases AVB, blurred vision, dryness of the mouth, skin flushes, acute urine retention or slowing of transit), anti-α-adrenergic effects (risk of sedation, of hypotension, of impotence), sympathetic reflex central inhibitor or else membrane-stabilizing effects (pro-arythmogenic effect). In particular, one of the formidable and feared effects of amitriptyline is QT prolongation that can lead to the death of a patient who has not been correctly monitored.

In particular, during oral administration of amitriptyline for the treatment of diabetic neuropathic pain, cases of sedation, of orthostatic hypotension and of anti-cholinergic effects have been reported (cf. in particular Kiani et al, Iran J Pharm. Res. 2015 Fall; 14(4):1263-8). In the long term, patients report memory problems, difficulties in concentration with considerable effects on the quality of their work or on their daily life.

Moreover, the efficacy of orally administered amitriptyline is slow (5 to 7 days of treatment are required in order to be able to begin to assess the efficacy of the product), variable according to patients and incomplete. It is consequently often necessary to use combinations of analgesics in order to overcome these drawbacks.

In addition, taking tricyclic antidepressants orally often has a bad reputation with patients because of their uses in various psychiatric disorders.

Given the problems of oral treatments, topical treatments have been attempted. The efficacy of amitriptyline administered topically for neuropathic pain has not been demonstrated. In particular, the article by Thompson et al., “Systematic review of topical amitriptyline for the treatment of neuropathic pain”, J. Clin. Pharm. Therm. 2015, 40, 496-503, concludes that controlled clinical trials reveal that topical amitriptyline is not efficacious in the treatment of neuropathic pain. The maximum dose used is 5% for a patient suffering from multiple sclerosis and exhibiting neuropathic pain. Likewise, the article “A phase III randomized, placebo-controlled study of topical amitriptyline and ketamine for chemotherapy-induced peripheral neuropathy”, Support Care Cancer, 2014 July; 22(7):1807-1814, concluded that a topical composition comprising 2% by weight of ketamine and 4% by weight of amitriptyline was not efficacious for treating post-chemotherapy neuropathic pain.

Thus, there is no satisfactory treatment for chemotherapy-induced neuropathic pain. Furthermore, treatments combining ketamine and amitriptyline, which appeared to give results in patients with post-shingles neuropathic pain or neuropathic pain of diabetic origin, have not made it possible to overcome chemotherapy-induced neuropathic pain, as noted in the abovementioned phase III clinical study.

Moreover, the doses envisaged, despite the disabling nature of this pain, have never exceeded 5%, whether orally or topically.

Moreover, patients suffering from neuropathies in the extremities (feet and hands) often exhibit damaged or even chapped and dried skin.

### DETAILED DESCRIPTION

While illustrative embodiments have been illustrated and described, it will be appreciated that various changes can be made therein without departing from the spirit and scope of the invention.

A subject of the invention is thus the provision of a composition based on amitriptyline which is efficacious when applied to the skin in the treatment of peripheral neuropathies and in particular of chemotherapy-induced neuropathies.

A subject of the invention is also a composition based on amitriptyline which makes it possible, in addition to overcoming neuropathic pain, to return to a healthier and more hydrated skin.

Other subjects of the invention will emerge on reading the description and the examples which follow.

It has been discovered, surprisingly, that a pharmaceutical composition for topical application comprising, in a pharmaceutically acceptable support suitable for topical application, at least 10% by weight of amitriptyline or of a pharmaceutically acceptable salt thereof, administered topically, makes it possible to efficaciously treat chemotherapy-induced peripheral neuropathic pain (or CIPN for chemotherapy-induced peripheral neuropathy).

A subject of the invention is thus a pharmaceutical composition comprising, in a pharmaceutically acceptable support suitable for topical application, from 10% to 30% by weight, relative to the total weight of the composition, of amitriptyline or of a pharmaceutically acceptable salt thereof, for use in the topical treatment of chemotherapy-induced peripheral neuropathic pain.

Topical application of the composition according to the invention is an efficacious treatment for chemotherapy-induced peripheral neuropathic pain.

Furthermore, the topical application of the composition according to the invention exhibits few, or even exhibits no, side effects. In particular, no skin irritation is observed at the site of application of the composition.

Amitriptyline has the formula (I) below:

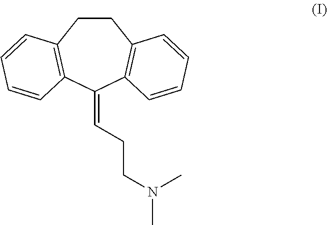 [SMILES: CN(C)CCC=C1c2ccccc2CCc2ccccc21]

In the context of the present invention, the term “pharmaceutically acceptable amitriptyline salt” is intended to mean the salts of amitriptyline compatible with a pharmaceutical composition, i.e. intended to be administered to human beings. In particular, the term “pharmaceutically acceptable amitriptyline salt” is intended to mean the hydrates, solvates, acid salts such as hydrochlorides and clathrates of amitriptyline.

Amitriptyline hydrochloride will be used as most particularly preferred amitriptyline salt.

As indicated above, the topical application of the composition according to the invention is an efficacious treatment for chemotherapy-induced peripheral neuropathic pain.

The application of the composition according to the invention, in the treatment of chemotherapy-induced neuropathic pain, has made it possible to obtain particularly spectacular results with regard to the results previously obtained. Thus, it has been possible to reduce pain classified according to the simple numerical scale between 4/10 and 7/10 according to patients to a value of virtually zero for all patients after 1 month of treatment.

The use of the composition according to the invention in the treatment of chemotherapy-induced neuropathic pain has made it possible to continue chemotherapy treatments, which would often have to be interrupted or suspended because of severe neuropathic pain.

A subject of the invention is thus also the use of the composition according to the invention in the context of a cancer treatment combining chemotherapy and treatment of neuropathic pain that may be chemotherapy induced. The composition according to the invention may thus be administered between chemotherapy regimens and may thus make it possible to continue the treatments.

The inventor has moreover discovered that the composition according to the invention can be applied preventively before a chemotherapy treatment and has, surprisingly, a neuroprotective effect, which would make it possible to reduce, or even prevent, chemotherapy-induced neuropathic pain.

The composition according to the invention can thus also be administered before beginning a chemotherapy treatment, the administration of the composition according to the invention being continued during and between the chemotherapy regimens and, if necessary, continued after the treatment depending on the state of neuropathic pain.

The composition according to the invention comprises from 10% to 30% by weight, preferably from 10% to 20% and in particular from more than 10% to 15% by weight of amitriptyline or of a pharmaceutically acceptable salt thereof relative to the total weight of the composition.

Particularly preferably, the amitriptyline is the only pharmaceutical active agent of the composition according to the invention.

In one preferred form, the composition contains the amitriptyline in the abovementioned proportions as sole pain-treating agent, in particular without any other analgesic agent or antidepressant or antiepileptic agent also sometimes recommended for the treatment of neuropathic pain, such as for example lidocaine, gabapentin, pregabalin, baclofen, capsaicin, ketamine.

This is particularly advantageous in the context of the invention since, contrary to the prior art, amitriptyline alone at a content of at least 10% by weight exhibits good efficacy in the treatment of chemotherapy-induced peripheral neuropathic pain.

It has also been noted that the application of the composition to skin explants in an ex vivo model results in a passage into the bloodstream of less than 0.1% relative to the amount of amitriptyline present in the composition. The very low passage into the bloodstream makes it possible to avoid the side effects noted for the treatments applied to neuropathies in the prior art. In particular, the bioconversion to nortriptyline is minor.

The pharmaceutical compositions according to the invention are generally in the form of an oil-in-water emulsion.

These compositions contain, as essential components, at least fatty substances, one or more hydrating active agents, non-ionic surfactants.

The oily phase of the composition according to the invention comprises one or more fatty substances.

The term “fatty substance” is intended to mean an organic compound which is water-insoluble at ambient temperature (25° C.) and at atmospheric pressure (1.013×10⁵ Pa) (solubility less than 5% by weight, and preferably less than 1% by weight, even more preferentially less than 0.1% by weight). They have in their structure at least one hydrocarbon-based chain comprising at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

Preferably, the fatty substance(s) are chosen from synthetic, animal, mineral or vegetable oils, silicone oils, fatty acids, fatty alcohols, waxes, gums and mixtures of these compounds.

As an example of a mineral oil, mention may be made of liquid paraffins of varied viscosities.

By way of vegetable oil, mention may in particular be made of sweet almond oil, palm oil, soybean oil, sesame oil and sunflower oil.

By way of animal oil, mention may in particular be made of lanolin, squalene, fish oil and mink oil.

By way of synthetic oil, mention may in particular be made of esters of alcohol and of fatty acid, such as cetearyl isononanoate, isopropyl palmitate and caprylic/caprylate triglycerides.

As an example of silicone oil, mention may in particular be made of dimethicone and cyclomethicone.

As an example of fatty acid, mention may in particular be made of stearic acid and palmitic acid.

As an example of fatty alcohol, mention may in particular be made of stearyl alcohol, cetostearyl alcohol and cetyl alcohol.

By way of wax, mention may in particular be made of beeswax (or cera alba), carnauba wax and candelilla wax.

By way of gum, mention may in particular be made of silicone gum.

Particularly preferably, the fatty substance(s) of the composition according to the invention are chosen from mineral oils, fatty acids, waxes and mixtures of these compounds.

Most particularly preferably, the composition according to the invention comprises a mixture of one or more mineral oils, of one or more fatty acids and of one or more waxes.

The fatty substance(s) preferably represent from 15% to 25% by weight, relative to the total weight of the composition, and in particular 20% to 25% by weight, relative to the total weight of the composition.

The composition according to the invention may also comprise one or more surfactants, which are preferably non-ionic, and which may or may not be oxyethylenated.

Particularly preferably, the composition according to the invention comprises one or more non-oxyethylenated non-ionic surfactants.

The compositions according to the invention may also contain glucolipid self-emulsifying systems, such as mixtures of fatty alcohol and of alkyl glycosides having 10 to 16 carbon atoms and in particular a mixture of cetylstearyl alcohol and cetearyl glucoside.

The non-ionic surfactant(s) can advantageously be chosen from sorbitan esters, glycerol esters, and mixtures of these compounds, polaxamers.

By way of sorbitan ester, mention may in particular be made of sorbitan stearate or sorbitan oleate.

By way of glycerol ester, mention may in particular be made of glyceryl stearate.

Preferably, the composition according to the invention comprises a mixture of one or more sorbitan esters and of one or more glycerol esters.

Advantageously, the surfactant(s) that can be used in the composition according to the invention represent, when they are present, from 2% to 8%, preferably 2% to 5% by weight, relative to the total weight of the composition.

The composition according to the invention may also comprise one or more gelling agents.

According to the invention, a gelling agent is any compound which, when added to a composition, increases the viscosity of said composition, the gelling agent representing from 0.01% to 4% by weight, preferably from 0.01% to 1% by weight, relative to the total weight of the composition.

By increasing the viscosity of the composition according to the invention, said composition is more stable over time.

The gelling agent(s) that can be used in the composition according to the invention are preferably chosen from carboxyvinyl polymers (carbomer), cellulose-based derivatives, xanthan gums, vegetable gums, aluminium/magnesium silicates, guar gums, polyacrylamide polymers, acrylate copolymers, modified starches, and mixtures of these compounds.

By way of carboxyvinyl polymer (carbomer), mention may in particular be made of Carbopol 981, Carbopol ETD 2020, Carbopol 980, Carbopol Ultrez 10 NF and Pemulen TR1, sold by Lubrizol.

By way of cellulose-based derivative, mention may in particular be made of hydroxypropylmethylcellulose and hydroxyethylcellulose.

By way of aluminium/magnesium silicate, mention may in particular be made of Veegum K and Veegum Ultra sold by Vanderbilt.

As polyacrylamide polymer, mention may in particular be made of the polyacrylamide/C13-14 isoparaffin/laureth-7 mixture, for example that sold by SEPPIC under the brand name Sepigel 305.

By way of modified starch, mention may in particular be made of Structures Solanace sold by Akzo Nobel.

Preferably according to the invention, the gelling agent(s) that can be used according to the invention are chosen from carboxyvinyl polymers (carbomer).

The gelling agent(s) that can be used in the composition according to the invention represent, when they are present, preferably from 0.1% to 4% by weight relative to the total weight of the composition.

The composition according to the invention advantageously comprises water.

In one preferred embodiment, the composition according to the invention comprises one or more hydrating active agents.

A hydrating active agent is an active agent capable of reducing the state of dryness of an epidermis.

Thus, the term “hydrating active agent” is intended to mean generally a compound which acts on the barrier function, with a view to maintaining the hydration of the stratum corneum, or an occlusive compound.

Mention may in particular be made of ceramides, sphingoid-based compounds, lecithins, glycosphingolipids, phospholipids, cholesterol and its derivatives, phytosterols (stigmasterol, β-sitosterol, campesterol), 1,2-diacylglycerol, 4-chromanone, pentacyclic triterpenes, glycosaminoglycans, sugars, polysaccharides, urea and glycerol.

Preferably, the hydrating active agent is glycerol.

Advantageously, the hydrating active agent(s) that can be used in the composition according to the invention represent, when they are present, from 7% to 15% by weight relative to the total weight of the composition.

The composition according to the invention may also comprise one or more additives or combinations of additives chosen from preservatives, stabilizers, flavour enhancers and pH adjusters.

As preservative, mention may in particular be made of phenoxyethanol.

Of course, those skilled in the art will choose the various additives or combinations of additives while taking great care to ensure that the properties intrinsically associated with the composition according to the invention are not impaired, or are barely impaired, by the envisaged additions.

The additives, when they are present in the composition according to the invention, generally each represent from 0.001% to 20% by weight relative to the total weight of the composition.

In one preferred embodiment of the invention, the composition comprises:
from 10% to 30%, preferably from 10% to 20% by weight, more preferentially from more than 10% to 15% by weight of amitriptyline or of a pharmaceutically acceptable salt thereof,
from 2% to 8% by weight of one or more non-ionic surfactants,
from 15% to 25% by weight of one or more fatty substances,
from 0.1% to 4% by weight of one or more gelling agents,
from 7% to 15% by weight of one or more hydrating active agents,
optionally from 0 to 3% by weight of one or more preservatives,
optionally from 0 to 1% by weight of one or more pH adjusters, so as to maintain the pH at around 7, in particular between 6.5 and 7.5,
water.

These compositions are particularly efficacious in the treatment of chemotherapy-induced neuropathic pain, since they make it possible not only to efficaciously treat the pain, but also to restore the skin that is often dehydrated at the painful extremities.

Preferably, the surfactant(s), the fatty substance(s), the gelling agent(s), the hydrating active agent(s), and the preservative(s) are as defined above.

Particularly preferably in this embodiment, the composition according to the invention comprises:
from 10% to 30%, preferably from 10% to 20% by weight, more preferentially from 10.5% to 15% by weight of amitriptyline or of a pharmaceutically acceptable salt thereof,
from 2% to 8% by weight of one or more surfactants chosen from sorbitan esters, glycerol esters, and mixtures of these compounds, or other surfactants allowing stabilization of the formula:
from 15% to 25% by weight of one or more fatty substances, including mineral oils, fatty acids, waxes and mixtures of these compounds,
from 0.1% to 4% by weight of one or more gelling agents including carboxyvinyl polymers,
from 7% to 15% by weight of one or more hydrating active agents,
optionally from 0 to 3% by weight of one or more preservatives,
optionally from 0 to 1% by weight of one or more pH adjusters,
water.

Most particularly preferably in this embodiment, the composition according to the invention comprises:
from 10% to 30%, preferably from 10% to 20% by weight, more preferentially from 10.5% to 15% by weight of amitriptyline or of a pharmaceutically acceptable salt thereof,
from 2% to 8% by weight of a mixture several non-ionic surfactants including one or more sorbitan esters and one or more glycerol esters,
from 15% to 25% by weight of a mixture of one or more mineral oils, of one or more fatty acids and of one or more waxes,
from 0.1% to 4% by weight of one or more carboxyvinyl polymers (carbomer),
from 7% to 15% by weight of glycerol,
optionally from 0 to 3% by weight of one or more preservatives,
optionally from 0 to 1% by weight of one or more pH adjusters,
water.

In particular, this embodiment makes it possible to reduce, or even eliminate, the side effects associated with the absorption of amitriptyline, in particular skin irritation at the site of application of the composition.

This embodiment also makes it possible to obtain good stability over time of the composition according to the invention at ambient temperature, but also at higher storage temperatures (45° C. for example).

Finally, this embodiment makes it possible advantageously to facilitate the penetration of the amitriptyline through the skin without passage into the bloodstream. The majority of the amitriptyline is concentrated in the dermis. Good therapeutic efficacy is thus obtained with good tolerance.

The pH of the compositions according to the invention is preferably between 5 and 8 and is adjusted with a base of NaOH or triethanolamine type.

The composition according to the invention is a topical composition.

The composition according to the invention may be in liquid, pasty or solid form, and more particularly in the form of salves, creams, milks, ointments. Preferably, the composition according to the invention is in the form of a light and unctuous cream.

The following examples illustrate the composition according to the invention and the advantages of this composition. However, they do not in any way represent a limitation of the present invention, but simply illustrate the invention.

### EXAMPLES

### Example 1

Composition in the Form of an Oil-In-Water Emulsion:

| | | |
|---|---|---|
| Amitriptyline | 10 | mg |
| Glyceryl stearate | 1 | mg |
| Other surfactants | 1 | mg |
| Paraffinum liquidum | 12 | mg |
| Palmitic acid | 1 | mg |
| Stearic acid | 1 | mg |
| Beeswax (cera alba) | 1 | mg |
| Carbomer | 0.1 | mg |
| Glycerol | 10 | mg |

| | |
|---|---|
| pH adjuster qs | pH 6.9 |
| Preservative | qs |
| Water | qs 100 mg |

The cream thus obtained was applied, once in the morning and evening, to the painful zones of a population of 31 patients with chemotherapy-induced peripheral neuropathic pain. It was applied to the hands and the feet.

Among the patients treated, three groups were distinguished according to how long the neuropathic pain had been present.

Group GR1 (11 patients) was treated within a period of one month after the occurrence of the neuropathic pain. These patients feel neuropathic pain of moderate intensity (visual analogue scale, VAS=4/10) of the type electrical discharges, burning, pins and needles. Because of the occurrence of this debilitating pain, the chemotherapy doses had to be decreased in the majority of cases, the chemotherapy had to be stopped for 2 patients. The application of the amitriptyline cream above for 3 to 5 days is efficacious at 100% and totally reduces the pain (VAS 0/10). The treatment is stopped, without recurrence, after one month making it possible to re-initiate the chemotherapy or to re-increase the doses.

Group GR2, the patients (13) feel neuropathic pain of the extremities of moderate to severe intensity (VAS of between 5 and 7/10) of the type burning, electrical discharges, pins and needles, sensation “of oedema” of the affected zones. The application of the amitriptyline cream at 10% is efficacious (VAS 2-3/10) after 15 days of treatment. At one month, the pain disappeared (VAS 0/10) in all the patients. The patients treated for chemotherapy-induced neuropathies continue the treatment throughout the chemotherapy regimens as a preventive measure.

Group GR3, the patients (7) feel neuropathic pain of severe intensity (VAS greater than 7/10) of the type burning, electrical discharges, stabbing, pins and needles, sensation “of oedema” in the affected zones. The functional consequence is major (difficulty gripping when the hands are affected, difficulty walking when the feet are affected, impossible to put on closed shoes, difficulty putting on clothing on the affected zone) and the patients present a depressive syndrome associated with neuropathic pain. For the latter patient group, the 10% amitriptyline cream begins to be efficacious (loss of 3 VAS points) starting from one month of treatment. The treatment is continued for 3 months (VAS less than 2/10).

### Example 2

Composition in the Form of an Oil-In-Water Emulsion:

| | | |
|---|---|---|
| Amitriptyline | 15 | mg |
| Glyceryl stearate | 1 | mg |
| Other surfactants | 1 | mg |
| Paraffinum liquidum | 12 | mg |
| Palmitic acid | 1 | mg |
| Stearic acid | 1 | mg |
| Beeswax (cera alba) | 1 | mg |
| Carbomer | 0.1 | mg |
| Glycerol | 10 | mg |

| | |
|---|---|
| pH adjuster qs | pH 6.9 |
| Preservative | qs |
| Water | qs 100 mg |

The composition in the form of a cream was administered to patients experiencing neuropathic pain of severe intensity (VAS of greater than 7/10) of the type burning, electrical discharges, stabbing, pins and needles, sensation “of oedema” in the affected zones. The administration of the cream provides very good pain control in one month of treatment (VAS between 0 and 2/10).

### Example 3

The cream of example 1 was applied, once in the morning and evening, to the painful zones of a population of 5 patients experiencing post-shingles peripheral neuropathic pain.

The patients feel neuropathic pain of the extremities of moderate to severe intensity (VAS between 6 and 8/10) of the type burning, electrical discharges, pins and needles, sensation “of oedema” of the affected zones. The application of the cream of example 1 to the thorax (4) and to the thighs (1) is efficacious (VAS 2-3/10) after 15 days of treatment. At one month, the pain disappeared (VAS 0/10) in all of the patients. The patients treated can stop the treatment.

### Example 4

The following cream was prepared in the form of an oil-in-water emulsion:

| | | |
|---|---|---|
| Amitriptyline | 10 | mg |
| Glyceryl stearate | 2 | mg |
| Sorbitan stearate | 1 | mg |
| Paraffinum liquidum | 8 | mg |
| Cetearyl ethylhexanoate | 5 | mg |
| Palmitic acid | 1 | mg |
| Stearic acid | 1 | mg |
| Beeswax (cera alba) | 2 | mg |
| Carbomer | 0.2 | mg |
| Hydroxyethyl acrylate/sodium | 0.1 | mg |
| acryloyldimethyl taurate copolymer | | |
| Glycerol | 10 | mg |

| | |
|---|---|
| pH adjuster qs | pH 6.9 |
| Preservative | qs |
| Water | qs 100 mg |

The cream was applied, once in the morning and evening, to the painful zones of a patient experiencing post-diabetic peripheral neuropathic pain.

The patient feels neuropathic pain of severe intensity (VAS of greater than 7/10) of the type burning, electrical discharges, stabbing, pins and needles, sensation “of oedema” in the affected zones. The functional consequence is major (difficulty gripping when the hands are affected, difficulty walking when the feet are affected, impossible to put on closed shoes, difficulty putting on clothing on the affected zone). The 10% amitriptyline cream begins to be efficacious (loss of 3 VAS points) starting from one month of treatment. The treatment is continued for 3 months (VAS less than 2/10).

### Example 5

2 patients suffering from colon cancer and having R-CHOP therapy regimens as chemotherapy, who had experienced, after the 1st therapy regimen, severe neuropathy in the hands of the type pins and needles, very intense painful cold sensation, VAS 7-8/10, were treated. Faced with the intensity of the neuropathy, the 2nd R-CHOP therapy regimen had been put back.

7 days before the 2nd therapy regimen, the patients applied the 10% amitriptyline cream to the hands and continued the application for 7 days after the chemotherapy regimen without suffering from neuropathy (VAS 1-2/10 without painful cold sensation).

### Example 6

Ex vivo study of the percutaneous amitriptyline absorption and of the presence of the pharmacologically active metabolite: nortriptyline.

A composition in the form of a cream containing 10% by weight of amitriptyline hydrochloride was applied to samples of human skin. The experiment was repeated 3 times with 3 samples of skin from 3 different donors, that is to say 9 samples. The skin samples are mounted in a Frantz cell and are brought to a surface temperature of 32° C.±1° C.

The formulation containing 10% by weight of amitriptyline hydrochloride by spreading it uniformly, using a spatula, on each skin sample in a proportion of 10 mg per cell, corresponding to 5 mg/cm² of skin.

The skin samples are rinsed 16 hours after application.

Each skin sample was placed on absorbent paper (dermis facing downwards) with tweezers.

The stratum corneum is removed using adhesive tapes.

After removal of the stratum corneum, the sample is perforated. The epidermis is then separated from the dermis. Each one of them is placed in separate vials.

The various samples were then extracted.

These tests made it possible to note that 90.6% to 98% of amitriptyline remains at the surface of the skin. Approximately 74% of the amitriptyline is present in the dermis compared with 26% in the epidermis.

The amitriptyline bioavailability is 22.5 μg.

A passage into the bloodstream of less than 0.1% and a very low bioconversion of the amitriptyline into nortriptyline of about 25 ng were observed.

1. A method of treatment of chemotherapy-induces peripheral neuropathic pain comprising applying a topical treatment comprising a pharmaceutical composition containing, in a pharmaceutically acceptable carrier suitable for topical application, from 10% to 30% by weight, relative to the total weight of the composition, of amitriptyline or a pharmaceutically acceptable salt thereof.

2. The method according to claim 1, wherein the pharmaceutical composition is applied onto peripheral parts.

3. The method according to claim 1, wherein the pharmaceutical composition comprises from 10% to 20% by weight, relative to the total weight of the composition, of amitriptyline or of a pharmaceutically acceptable salt thereof.

4. The method according to claim 1, wherein the pharmaceutical composition comprises from 10% to 15% by weight, relative to the total weight of the composition, of amitriptyline or of a pharmaceutically acceptable salt thereof.

5. The method according to claim 1, wherein amitriptyline is a sole agent for treating neuropathic pain.

6. The method according to claim 1, wherein the method further comprises applying the pharmaceutical composition before a chemotherapy treatment to reduce or prevent chemotherapy-induced peripheral neuropathic pain.

7. The method according to claim 1, wherein the method further comprises applying the pharmaceutical composition between two chemotherapy sessions to reduce or prevent chemotherapy-induced neuropathic pain and other neuropathic pain.

8. The method according to claim 1, wherein the method further comprises:
applying the pharmaceutical composition before a chemotherapy session, wherein the chemotherapy session comprises two or more chemotherapy regimens;
applying the pharmaceutical composition between and during the two or more chemotherapy regimens; and
applying the pharmaceutical composition after the chemotherapy session, based on a state of chemotherapy-induced neuropathic pain.

9. The method of claim 1, wherein the pharmaceutical composition is in the form of an oil-in-water emulsion and comprises at least fatty substances, one or more hydrating active agents, and non-ionic surfactants.

10. The method of claim 1, wherein the pharmaceutical composition comprises one or more fatty substances.

11. The method according to claim 10, wherein the fatty substances are chosen from synthetic, animal, mineral or vegetable oils, silicone oils, fatty acids, fatty alcohols, waxes, gums, and mixtures thereof.

12. The method according to claim 1, wherein the pharmaceutical composition comprises one or more non-ionic surfactants chosen from sorbitan esters, glycerol esters, and mixtures thereof.

13. The method according to claim 1, wherein the pharmaceutical composition comprises one or more gelling agents chosen from carboxyvinyl polymers, cellulose-based derivatives, xanthan gums, vegetable gums, aluminum/magnesium silicates, guar gums, polyacrylamide polymers, acrylate copolymers, modified starches, and mixtures thereof.

14. The method according to claim 1, wherein the pharmaceutical composition comprises:
from 10% to 30% by weight of amitriptyline or of a pharmaceutically acceptable salt thereof,
from 2% to 8% by weight of one or more surfactants,
from 15% to 25% by weight of one or more fatty substances,
from 0.1% to 4% by weight of one or more gelling agents,
from 7% to 15% by weight of one or more hydrating active agents, and
water.

15. The method according to claim 14, wherein the pharmaceutical composition further comprises:
from 10% to 30% by weight of amitriptyline or of a pharmaceutically acceptable salt thereof,
from 2% to 8% by weight of one or more surfactants chosen from sorbitan esters, glycerol esters, and mixtures of these compounds,
from 15% to 25% by weight of one or more fatty substances chosen from mineral oils, fatty acids, waxes and mixtures of these compounds,
from 0.1% to 4% by weight of one or more gelling agents chosen from carboxyvinyl polymers,
from 7% to 15% by weight of one or more hydrating active agents, and
water.

16. The method according to claim 14, wherein the pharmaceutical composition further comprises:
from 10% to 30% by weight of amitriptyline or of a pharmaceutically acceptable salt thereof,
from 2% to 8% by weight of a mixture of one or more sorbitan esters and of one or more glycerol esters,
from 15% to 25% by weight of a mixture of one or more mineral oils, of one or more fatty acids and of one or more waxes,
from 0.1% to 4% by weight of one or more carboxyvinyl polymers,
from 7% to 15% by weight of glycerol, and
water.

17. The method of claim 14, wherein the pharmaceutical composition further comprises from 0% to 3% by weight of one or more preservatives.

18. The method of claim 14, wherein the pharmaceutical composition further comprises from 0% to 1% by weight of one or more pH adjusters.

19. The method according to claim 1, wherein the pharmaceutical composition is in the form of a cream.
